(19) United States
(12) Patent Application Publication
(10) Pub. No.: US 2011/0087254 A1
(43) Pub. Date: Apr. 14, 2011

(54) ROTATIONAL ATHERECTOMY DEVICE WITH KEYED EXCHANGEABLE DRIVE SHAFT

(75) Inventor: Ryan D. Welty, Blaine, MN (US)

(73) Assignee: CARDIOVASCULAR SYSTEMS, INC., St. Paul, MN (US)

(21) Appl. No.: 12/576,601

(22) Filed: Oct. 9, 2009

(51) Int. Cl. A61B 17/22

(52) U.S. Cl. 606/159

(57) ABSTRACT

An atherectomy device with an exchangeable drive shaft is disclosed, having a mechanical coupling that can allow for axial translation of the drive shaft while keeping the drive shaft rotationally locked to the prime mover. The coupling is geometrically keyed, with one side of the coupling having an aperture with a particular internal cross-section, and the other side of the coupling having an external cross-section that matches all or a part of the corresponding internal cross-section. Key shapes may be hexagonal, square, n-sided polygonal, star-shaped, or any other suitable shape. The keys may optionally include one or more rounded corners to simplify manufacturing. Axial motion may be locked by an optional twist-lock connection of two elements that surround the keyed coupling.

### CROSS-REFERENCE TO RELATED APPLICATIONS

Not Applicable

### STATEMENT REGARDING FEDERALLY SPONSORED RESEARCH OR DEVELOPMENT

Not Applicable

### BACKGROUND OF THE INVENTION

1. Field of the Invention

The invention relates to devices and methods for removing tissue from body passageways, such as removal of atherosclerotic plaque from arteries, utilizing a rotational atherectomy device. In particular, the invention relates to improvements in a rotational atherectomy device having an exchangeable drive shaft.

2. Description of the Related Art

A variety of techniques and instruments have been developed for use in the removal or repair of tissue in arteries and similar body passageways. A frequent objective of such techniques and instruments is the removal of atherosclerotic plaque in a patient's arteries. Atherosclerosis is characterized by the buildup of fatty deposits (atheromas) in the intimal layer (i.e., under the endothelium) of a patient's blood vessels. Very often over time what initially is deposited as relatively soft, cholesterol-rich atheromatous material hardens into a calcified atherosclerotic plaque. Such atheromas restrict the flow of blood, and therefore often are referred to as stenotic lesions or stenoses, the blocking material being referred to as stenotic material. If left untreated, such stenoses can cause angina, hypertension, myocardial infarction, strokes and the like.

Several kinds of atherectomy devices have been developed for attempting to remove some or all of such stenotic material. In one type of device, such as that shown in U.S. Pat. No. 4,990,134 (Auth), a rotating burr covered with an abrasive cutting material, such as diamond grit (diamond particles or dust), is carried at the distal end of a flexible, rotatable drive shaft.

U.S. Pat. No. 5,314,438 (Shturman) shows another atherectomy device having a rotatable drive shaft with a section of the drive shaft having an enlarged diameter, at least a segment of this enlarged diameter section being covered with an abrasive material to define an abrasive segment of the drive shaft. When rotated at high speeds, the abrasive segment is capable of removing stenotic tissue from an artery.

U.S. Pat. No. 5,314,407 (Auth) shows details of a type of handle which may be used in conjunction with rotational atherectomy devices of the type shown in the Auth '134 and Shturman '438 patents. A handle of the type shown in the Auth '407 patent has been commercialized by Heart Technology, Inc. (Redmond, Wash.), now owned by Boston Scientific Corporation (Natick, Mass.) in the rotational atherectomy device sold under the trademark Rotablator®. The handle of the Rotablator® device includes a variety of components, including a compressed gas driven turbine, a mechanism for clamping a guide wire extending through the drive shaft, portions of a fiber optic tachometer, and a pump for pumping saline through the drive shaft.

The connection between the drive shaft (with its associated burr) and the turbine in the Rotablator® device is permanent; yet, frequently it is necessary to use more than one size burr during an atherectomy procedure. That is, often a smaller size burr is first used to open a stenosis to a certain diameter, and then one or more larger size burrs are used to open the stenosis further. Such use of multiple burrs of subsequently larger diameter is sometimes referred to as a “step up technique” and is recommended by the manufacturer of the Rotablator® device. In the multiple burr technique it is necessary to use a new Rotablator® device for each such successive size burr. Accordingly, there is a need for an atherectomy system that would permit a physician to use only one handle throughout an entire procedure and to attach to such handle an appropriate drive shaft and tissue removing implement (e.g., a burr) to initiate the procedure and then exchange the drive shaft and the tissue removing implement for a drive shaft having a tissue removing implement of a different size or even a different design.

A subsequent version of the Rotablator® has been introduced with the ability to exchange a flexible distal portion of the drive shaft together with a burr for another distal portion of a drive shaft having a different size burr. Technical details of such a system are contained in U.S. Pat. No. 5,766,190, titled “Connectable driveshaft system”, and issued on Jun. 16, 1998 to Wulfman. This system utilizes a flexible drive shaft having a connect/disconnect feature allowing the physician to disconnect the exchangeable distal portion of the flexible drive shaft together with the burr from the flexible proximal portion of the drive shaft which is connected to the turbine of the handle, thus permitting the burr size to be changed without discarding the entire atherectomy unit. Each exchangeable drive shaft portion is disposed within its own exchangeable catheter and catheter housing. The flexible proximal portion of the drive shaft in this system is permanently attached to the turbine and is not exchanged. This system has been commercialized by Boston Scientific under the trademark Rotalink System®. While the Rotalink System® does permit one to change the burr size, the steps required to actually disconnect the exchangeable portion of the drive shaft and replace it with another exchangeable portion of the drive shaft are quite involved and require relatively intricate manipulation of very small components.

First, a catheter housing must be disconnected from the handle and moved distally away from the handle to expose portions of both the proximal and distal sections of the flexible drive shaft which contain a disconnectable coupling. This coupling is disconnected by sliding a lock tube distally, permitting complementary lock teeth on the proximal and distal portions of the flexible drive shaft to be disengaged from each other. A similar flexible distal drive shaft portion with a different burr may then be connected to the flexible proximal portion of the drive shaft. To accomplish such assembly, the lock tooth on the proximal end of the distal replacement portion of the drive shaft must first be both longitudinally and rotationally aligned with the complementary lock tooth at the distal end of the proximal portion of the drive shaft. Since the flexible drive shaft typically is less than 1 mm in diameter, the lock teeth are similarly quite small in size, requiring not insignificant manual dexterity and visual acuity to properly align and interlock the lock teeth. Once the lock teeth have been properly interlocked with each other, the lock tube (also having a very small diameter) is slid proximally to secure the coupling. The catheter housing must then be connected to the handle housing.

While this system does permit one to exchange one size burr (together with a portion of the drive shaft) for a burr of another size, the exchange procedure is not an easy one and must be performed with considerable care. The individual performing the exchange procedure must do so while wearing surgical gloves to protect the individual from the blood of the patient and to maintain the sterility of the elements of the system. Surgical gloves diminish the tactile sensations of the individual performing the exchange procedure and therefore make such exchange procedure even more difficult.

In recent years, there has been an effort to develop an atherectomy device with easier attachment and/or exchange of the drive shaft and its tissue removing implement.

For instance, four exemplary patents are U.S. Pat. Nos. 6,024,749, 6,077,282, 6,129,734 and 6,852,118, all issued to Shturman et al, and all incorporated by reference in their entirety herein. Collectively, these four patents disclose an atherectomy device having an exchangeable drive shaft cartridge comprising a housing that is removably attachable to the device's handle housing. The exchangeable cartridge includes a longitudinally movable tube that is removably attached to the prime mover carriage and a rotatable drive shaft that is removably attachable to the prime mover. A coupling is provided which connects the longitudinally extendible tube to the prime mover while indexing the relative position of the longitudinally extendible tube and the proximal portion of the drive shaft.

For devices in which the drive shaft is fixedly attached to the prime mover by a frictional fit, there may be instances where the frictional fit does not have sufficient strength to maintain contact. For instance, if the abrasive head contacts an unusually hard part of the blockage, there may be a “kick” transmitted from the distal end to the proximal end of the drive shaft as a torque. The “kick” may have a sufficient force to knock loose the frictional fit, resulting in a malfunctioning of the device. This is unacceptable.

Accordingly, there exists a need for an atherectomy device with an exchangeable drive shaft, where the drive shaft is locked with sufficient rotational strength to the prime mover.

### BRIEF SUMMARY OF THE INVENTION

An embodiment is a rotational atherectomy device, comprising: a handle housing; an elongated, flexible, rotatable drive shaft having a proximal end at the handle housing and a distal end opposite the proximal end for insertion into a vasculature of a patient; a drive shaft coupler fixedly attached to the proximal end of the drive shaft and facing away from the drive shaft; a prime mover within the handle housing for rotating the drive shaft; and a prime mover coupler rotatably coupled to the prime mover and facing the drive shaft. The drive shaft coupler and the prime mover coupler have engageable lateral cross-sections that are complementary and are geometrically keyed. Engagement of the complementary lateral cross-sections allows axial translation between the drive shaft coupler and the prime mover coupler and prohibits rotational motion between the drive shaft coupler and the prime mover coupler.

Another embodiment is a method for removing a blockage from a vessel, comprising: inserting a guide wire into a vasculature of a patient; advancing the guide wire through the vasculature to the blockage; removably attaching a proximal end of a first drive shaft to a prime mover, the attachment rotatably securing the first drive shaft to the prime mover and allowing longitudinal translation between the first drive shaft and the prime mover; advancing the first drive shaft over the guide wire until a first abrasive head at a distal end of the first drive shaft is proximate the blockage; rotating the first drive shaft; partially removing the blockage in the vessel, the removal comprising a size determined by the first abrasive head; and retracting the first drive shaft from the vasculature of the patient.

### BRIEF DESCRIPTION OF THE SEVERAL VIEWS OF THE DRAWINGS

FIG. 1 is a perspective view of a rotational atherectomy device of the prior art.

FIG. 2 is an enlarged perspective, partially broken-away view of a portion of the device shown in FIG. 1, illustrating an exchangeable drive shaft cartridge connected to the handle housing, according to the prior art.

FIG. 3 is a broken away, longitudinal cross-section of the atherectomy device shown in FIG. 2.

FIG. 4 is a longitudinal cross-sectional view of FIG. 3, taken along lines A-A thereof; and illustrating a flexible fluid supply tube attached to the exchangeable drive shaft cartridge.

FIG. 5 is a schematic cross-sectional drawing of an atherectomy device, in which the replaceable drive shaft is removed from the prime mover.

FIG. 6 is a schematic cross-sectional drawing of the atherectomy device of FIG. 5, in which the replaceable drive shaft is attached to the prime mover.

FIG. 7 is a lateral cross-sectional drawing of the couplers of FIG. 6, taken along line A-A, in which the complementary shape is an equilateral triangle.

FIG. 8 is a lateral cross-sectional drawing of the couplers of FIG. 6, taken along line A-A, in which the complementary shape is a square.

FIG. 9 is a lateral cross-sectional drawing of the couplers of FIG. 6, taken along line A-A, in which the complementary shape is a regular pentagon.

FIG. 10 is a lateral cross-sectional drawing of the couplers of FIG. 6, taken along line A-A, in which the complementary shape is a hexagon.

FIG. 11 is a lateral cross-sectional drawing of the couplers of FIG. 6, taken along line A-A, in which the complementary shape is an octagon.

FIG. 12 is a lateral cross-sectional drawing of the couplers of FIG. 6, taken along line A-A, in which the complementary shape is a trapezoid.

FIG. 13 is a lateral cross-sectional drawing of the couplers of FIG. 6, taken along line A-A, in which the complementary shape is a cross.

FIG. 14 is a lateral cross-sectional drawing of the couplers of FIG. 6, taken along line A-A, in which the complementary shape is a right triangle.

FIG. 15 is a lateral cross-sectional drawing of the couplers of FIG. 6, taken along line A-A, in which the complementary shape is a parallelogram.

FIG. 16 is a lateral cross-sectional drawing of the couplers of FIG. 6, taken along line A-A, in which the complementary shape is a square with rounded corners.

FIG. 17 is a cross-sectional schematic drawing of an atherectomy device, in which the longitudinal position of the drive shaft is manually controllable.

### DETAILED DESCRIPTION OF THE INVENTION

An atherectomy device with an exchangeable drive shaft is disclosed, having a mechanical coupling that can allow for axial translation of the drive shaft while keeping the drive shaft rotationally locked to the prime mover. The coupling is geometrically keyed, with one side of the coupling having an aperture with a particular internal cross-section, and the other side of the coupling having an external cross-section that matches all or a part of the corresponding internal cross-section. Key shapes may be hexagonal, square, n-sided polygonal, star-shaped, or any other suitable shape. The keys may optionally include one or more rounded corners to simplify manufacturing. Axial motion may be locked by an optional twist-lock connection of two elements that surround the keyed coupling.

The preceding paragraph is merely a summary, and should not be construed as limiting in any way. A more detailed description follows.

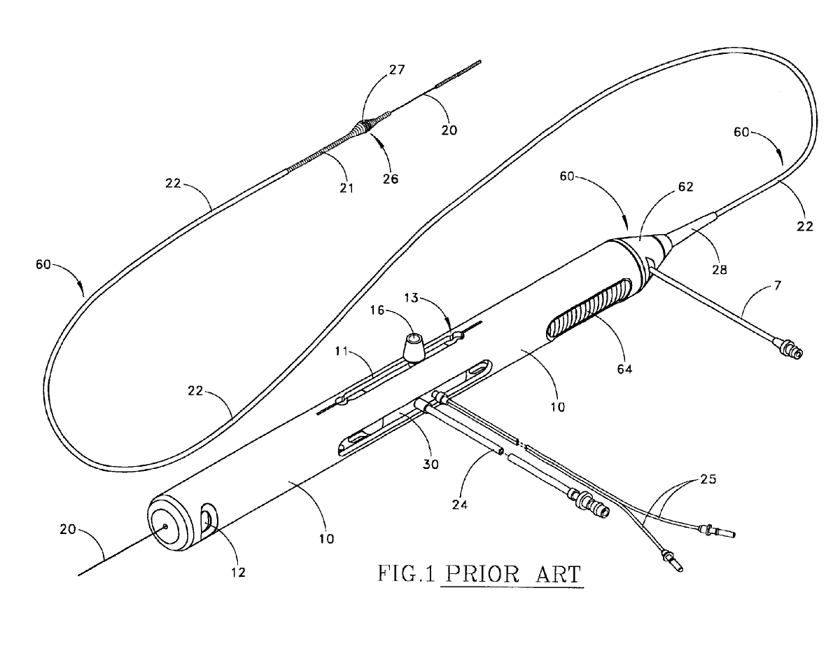

FIG. 1 illustrates a known rotational atherectomy device having an exchangeable drive shaft cartridge. The device desirably includes a tubular handle housing 10. The handle housing 10 has a proximal portion which carries a guide wire clamp or brake mechanism 12, an intermediate portion which carries a prime mover carriage 30, and a distal portion which is adapted to releasably interlock with an exchangeable drive shaft cartridge 60. The details of this system are described in U.S. Pat. Nos. 6,024,749, 6,077,282 and 6,852,118, all of which issued to Shturman, the disclosures of which are incorporated herein in their entirety by reference.

The prime mover carriage 30 can be moved longitudinally within the handle housing 10 through a limited range of motion. A control knob 16 (operatively secured to the prime mover carriage 30) is provided to facilitate advancing and retracting the prime mover carriage 30 with respect to the handle housing 10. This allows the distal end of the drive shaft to be moved within its range of operating positions.

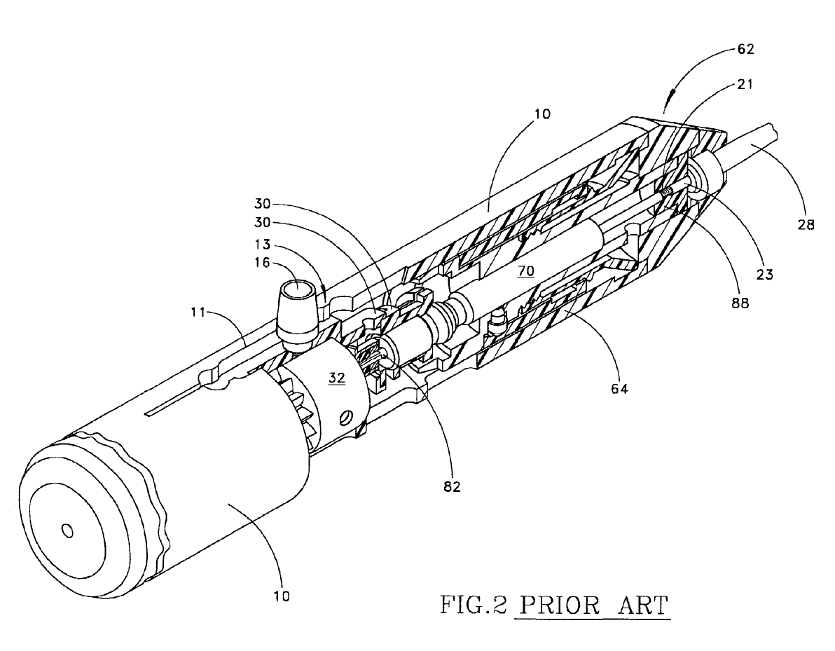

The prime mover carriage 30 carries a prime mover 32. The prime mover 32 is shown in FIGS. 2-4. Preferably the prime mover is a compressed gas driven turbine. The turbine may be powered by, for instance, compressed nitrogen or compressed air. For this purpose a compressed gas supply line 24 may be provided, the supply line 24 being connected to the prime mover carriage 30. A pair of fiber optic cables 25 may also be provided for monitoring the speed of rotation of the turbine (for instance, as described in the Auth '407 patent and implemented in the Rotablator® device).

The exchangeable drive shaft cartridge 60 includes a cartridge housing 62, an elongated catheter 22 extending distally from the cartridge housing 62, a rotatable flexible drive shaft 21 disposed within the catheter 22, a longitudinally movable slide 64, and a longitudinally movable tube 70 carried within the cartridge housing 62. The longitudinally movable tube 70 and other components are discussed below in connection with FIGS. 2-4. The elongated catheter 22 is carried by the cartridge housing 62 and has a proximal end portion which is disposed within a short rigid tube 23. The rigid tube 23 is secured within a generally tubular end piece 88 of the cartridge housing 62. Preferably a strain relief element 28 is disposed around the distal portion of the rigid tube 23 and the proximal portion of the catheter 22. The strain relief element 28 also is secured to the cartridge housing 62.

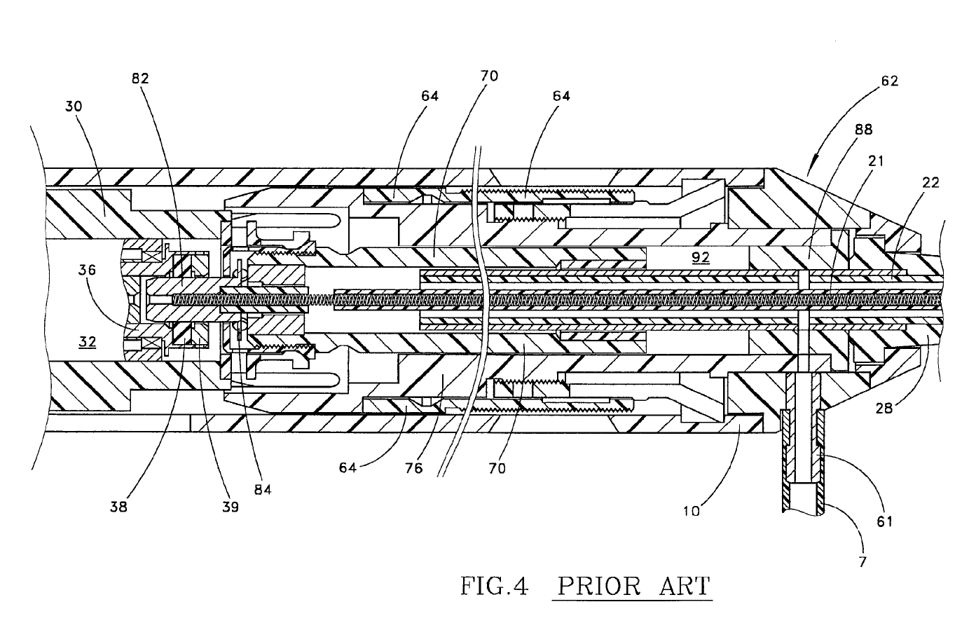

The exchangeable drive shaft cartridge 60 includes a flexible fluid supply tube 7. One end of the fluid supply tube 7 communicates with an external fluid supply (not shown) while the other end of the tube 7 is attached to a rigid fitting 61 of the cartridge housing 62. The flexible fluid supply tube 7 is in fluid communication with the inner lumen of the catheter 22 (see, for instance, FIG. 4), supplying fluid to help reduce friction between the rotating drive shaft 21 and non-rotating elements disposed within (for instance, the guide wire 20) and around the drive shaft 21.

The flexible drive shaft 21 is rotatable over a guide wire 20 and includes a proximal portion, an intermediate portion, and a distal portion. The proximal portion of the drive shaft 21 is removably attachable to the prime mover. This portion of the drive shaft is not visible in FIG. 1. The intermediate portion of the drive shaft 21 is disposed primarily within the catheter 22 and therefore also is not visible in FIG. 1. The distal portion of the drive shaft 21 extends distally from the catheter 22 and includes a tissue removal implement 26. The tissue removal implement 26 in the illustrated embodiment includes an eccentric enlarged diameter section of the drive shaft 21. A portion of the eccentric enlarged diameter section is covered with an abrasive material to define an abrasive segment 27 of the drive shaft 21. The diamond-coated burr attached at the distal end of the drive shaft and described by Auth in U.S. Pat. No. 4,990,134 may also be used. It should be understood that any suitable tissue removal implement may be used.

By comparing FIG. 1 with FIG. 2 one can see that the structure in FIG. 2 is not quite to scale with respect to FIG. 1. For example, the slot 11 is considerably shortened in FIG. 2 with respect to FIG. 1. In many other drawings (particularly longitudinal cross-sections) the diameter of the device and its components, as well as wall thickness, have been exaggerated so that the structural details of the device can be more clearly depicted and understood. The atherectomy device depicted in FIG. 1 is generally to scale, except for the length of the catheter 22 and drive shaft 21, which are actually substantially longer. Deviations from scale in the drawings should be readily apparent to one of ordinary skill in the art.

A drive shaft attachment mechanism is provided to removably attach the drive shaft 21 to the prime mover. The drive shaft attachment mechanism includes a prime mover socket 38 carried by the hollow prime mover 36, and an elongated shank 82 carried by the proximal end portion of the drive shaft 21. The drive shaft shank 82 is removably insertable into the prime mover socket 38. Preferably at least one of the drive shaft shank 82 and the prime mover socket 38 is radially resilient. In the preferred design shown in the drawings, the prime mover socket 38 is resilient. The prime mover socket 38 may be made to be radially resilient in a variety of ways. In the drawings the prime mover socket 38 consists of a resilient collar secured inside a recess in the hollow turbine shaft 36 by a cap 39. A variety of other suitable ways may also be utilized to secure a prime mover socket 38 to the turbine shaft 36.

The inner diameter of the prime mover socket 38 is selected to provide a sufficiently tight interference fit with the drive shaft shank 82 so that, when the drive shaft 21 is attached to the prime mover, the shank 82 and the drive shaft 21 will both rotate and move longitudinally together with the prime mover socket 38 and the prime mover when the prime mover is rotated or moved longitudinally with respect to the handle housing 10.

The elongated shank 82 is secured, either directly or indirectly, to the proximal end portion of the flexible drive shaft 21. Suitable adhesives or other conventional attachment methods may be utilized to attach the shank 82 to the flexible drive shaft 21. Moreover, the proximal end portion of the drive shaft 21 can itself constitute the shank if it is constructed in such a fashion as to be removably insertable into the prime mover socket 38.

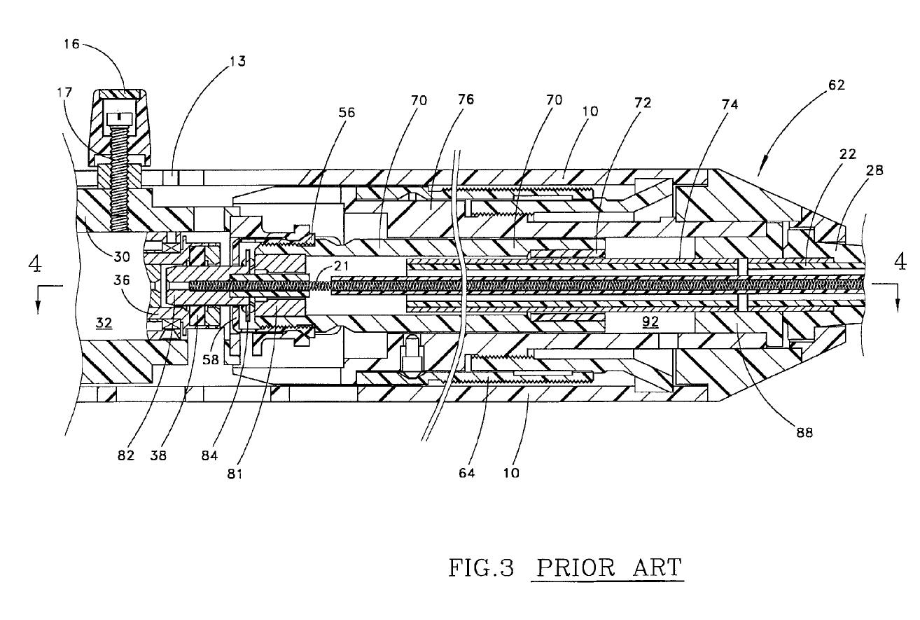

The elongated shank 82 preferably includes proximal and distal portions. A substantial length of the proximal portion is removably insertable into the prime mover socket 38, while the distal portion preferably includes a radially outwardly extending flange 84. As shown in FIGS. 3-4, the flange 84 is positioned between (and spaced away from) proximal and distal abutment surfaces associated with the proximal end portion of the longitudinally movable tube 70. The flange 84 abuts the distal abutment surface associated with the longitudinally movable tube 70 when the shank 82 is inserted into the prime mover socket 38. The flange 84 abuts the proximal abutment surface associated with the longitudinally movable tube 70 when the shank 82 is pulled out of the prime mover socket 38. The distal abutment surface associated with the tube 70 in this embodiment is formed by bushing 81 and/or the tube 70 itself. The proximal abutment surface associated with the tube 70 is formed by a flange 58 of the collar 56 carried by (and forming a distal end of) the longitudinally movable tube 70.

The longitudinally movable tube 70 is carried within the tubular core 76 of the cartridge housing 62 and has a proximal end portion which is removably attachable to the prime mover carriage 30 for longitudinal movement therewith. The longitudinally movable tube 70 surrounds a length of the flexible drive shaft 21 and facilitates longitudinal movement of the drive shaft 21 (together with the prime mover) with respect to the handle housing 10, the cartridge housing 62 and the catheter 22.

The longitudinally movable tube 70 is slidably received in an elongated annular space 92 defined within the tubular core 76 of the cartridge housing 62. The movable tube 70 is longitudinally moveable within that annular space 92 with respect to the cartridge housing 62. Desirably at least a portion of the inner surface of the longitudinally movable tube 70 is provided with a low-friction lining 72. The lining 72 helps minimize friction between the movable tube 70 and the stationary tube 74 as the longitudinally movable tube 70 is moved proximally and distally. The lining 72 may be made from any suitable material, such as polytetrafluoroethylene tubing. If so desired, the lining may be omitted and the movable tube 70 itself may be made of a low friction material.

The atherectomy device also includes a tube attachment mechanism positioned to removably attach the longitudinally movable tube 70 to the prime mover carriage 30. The tube attachment mechanism, as shown in FIGS. 2-4, includes a resilient positioning mechanism for moving the prime mover carriage 30 and the shank 82 proximally with respect to the longitudinally movable tube 70 after the longitudinally movable tube 70 has been attached to the prime mover carriage 30 and after the prime mover carriage 30 has been moved to its range of working positions (for instance, the control knob 16 and its shaft 17 have been moved proximally through the narrowed segment 13). The resilient positioning mechanism spaces the flange 84 of the shank 82 away from both distal and proximal abutment surfaces associated with the longitudinally movable tube 70 to permit free rotation of the shank 82 with respect to the longitudinally movable tube 70.

Examples of such attachment mechanisms are shown in U.S. Pat. Nos. 6,077,282, 6,024,749 and 6,852,118 (all issued to Shturman and cited above).

Having reviewed an exemplary known atherectomy device in FIGS. 1-4, we note that many aspects of this known device may be used with the present design. For instance, the construction of the drive shaft, the abrasive head at the distal end of the drive shaft, the plumbing of gases and fluids in the handle and catheter, the control electronics for monitoring and adjusting rotational speed, and so forth, may all be carried over from the known device disclosed in U.S. Pat. No. 6,852,118, or from any other known atherectomy device. All of these aspects may be used with the present design.

We now turn our attention to the proximal end of the drive shaft. Specifically, we examine the connection between the drive shaft and the prime mover.

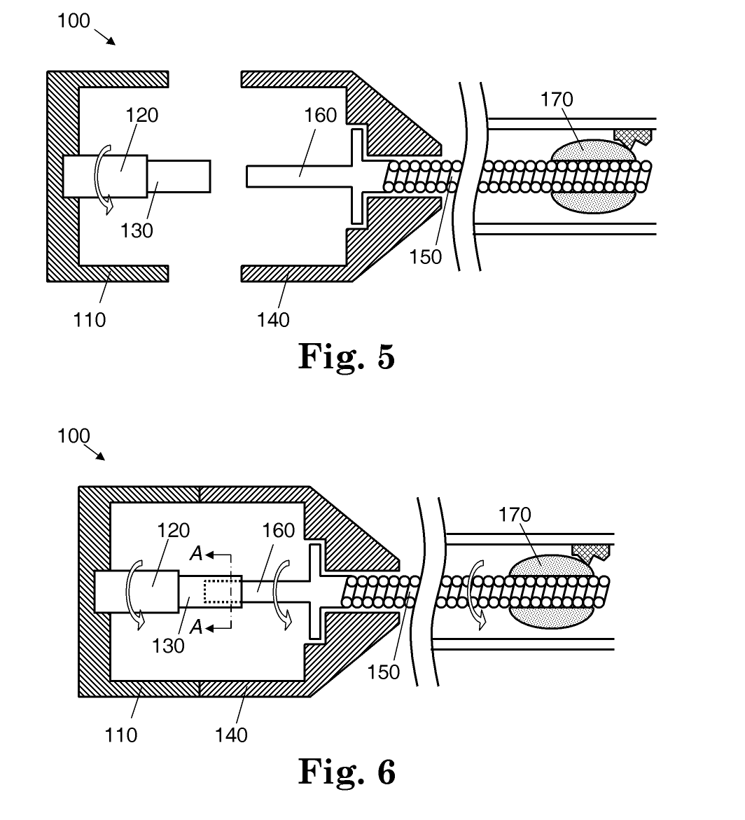

FIG. 5 is a schematic cross-sectional drawing of an atherectomy device, in which the replaceable drive shaft 150 is removed from the prime mover 120. The drive shaft 150 extends into the vasculature of a patient, and an abrasive head 170 at the distal end of the drive shaft 150 is rotated, along with the drive shaft itself, to remove all or a part of a blockage in the blood vessel.

FIG. 5 is a very basic schematic drawing, and for clarity omits most or all of the elements that are not directly involved with the coupling between the drive shaft 150 and the prime mover 120. For instance, the guide wire, which is an important element for operation of the atherectomy device, is not shown in FIG. 5 or in subsequent drawings. In addition, the coupling may itself be longitudinally translatable, so that the drive shaft may be advanced or retracted with respect to the guide wire and/or the catheter, independent of the coupling. It will be understood that such omitted elements may be similar in construction and function to those in FIGS. 1-4 or in the above-referenced U.S. patents.

In FIG. 5, the atherectomy device 100 includes a fixed handle portion 110 and an exchangeable handle portion 140 that attaches to the fixed handle portion 110. In some cases, the fixed and exchangeable handle portions are both cylindrical in lateral cross section. In other cases, the fixed and exchangeable handle portions have non-cylindrical cross-sections, such as square, rectangular, hexagonal, or any other suitable shape.

The attachment between the handle portions may be done in one of any number of known ways. For instance, the handle portions 110 and 140 may each include a set of mated threads, so that the handle portions may be screwed together. Alternatively, the handle portions 110 and 140 may include nested cylinders that lock together by a twist mechanism. In general, it is preferable that the fixed and exchangeable handle portions attach together in a way that provides at least a rough alignment for the drive shaft 150 and the prime mover 120.

The drive shaft 150 and the prime mover 120 are rotatably connectable by a pair of matched elements, namely the prime mover coupler 130 attached to (or made integral with) the prime mover 120 and the drive shaft coupler 160 attached to (such as, by a laser butt weld, or alternatively made integral with) the drive shaft 130.

The prime mover coupler 130 and the drive shaft coupler 160 are geometrically keyed to each other, so that a portion of one fits inside a portion of the other. When the two couplers are attached, they are free to longitudinally translate with respect to each other, but are preventing from rotating with respect to each other.

When the handle portions 110 and 140 are brought together, as in FIG. 6, a portion of the drive shaft coupler 160 fits inside a portion of the prime mover coupler 130. In the portion of overlap, the external profile of the drive shaft coupler fits completely within the internal profile of the aperture in the prime mover coupler. Furthermore, the matched external and internal profiles of the couplers are chosen so that when engaged, the couplers are prohibited from rotating with respect to each other.

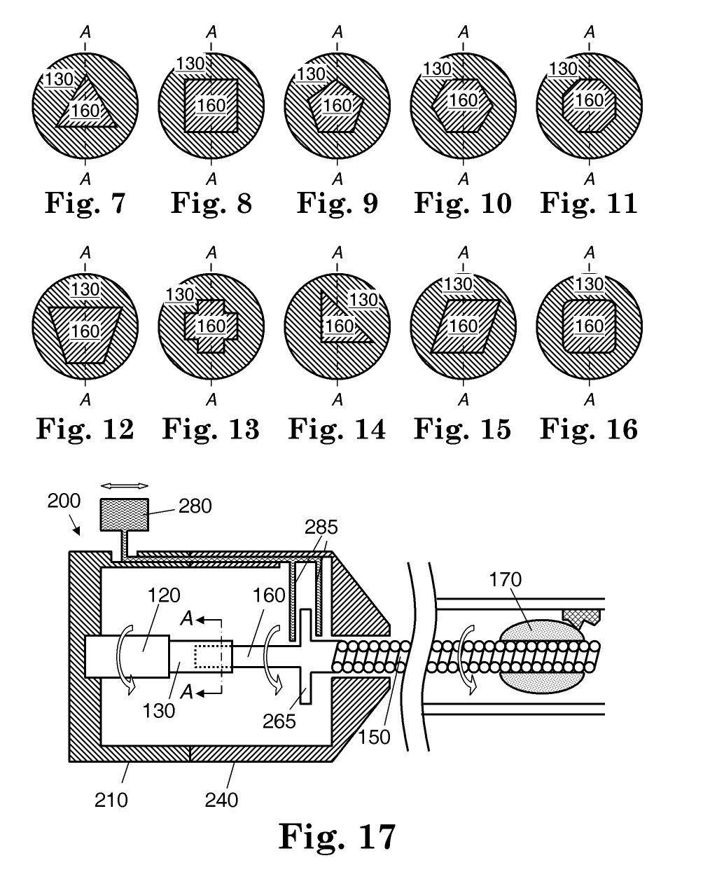

Exemplary complementary lateral cross-sections of the couplers, shown as line A-A in FIG. 6, are shown in FIGS. 7-16.

In FIGS. 7-16, the prime mover coupler 130 is shown having an aperture with a generally polygonal shape, such as an equilateral triangle (FIG. 7), a square (FIG. 8), a regular pentagon (FIG. 9), a hexagon (FIG. 10), an octagon (FIG. 11), a trapezoid (FIG. 12), a cross (FIG. 13), a right triangle (FIG. 14), a parallelogram (FIG. 15) and a square with rounded corners (FIG. 16).

In many of these designs, there is a symmetry that allows one coupler to be inserted into the other in multiple orientations. For instance, the parallelogram (FIG. 15) has two-fold symmetry, the equilateral triangle (FIG. 7) has three-fold symmetry, the square (FIG. 8), cross (FIG. 13), and square with rounded corners (FIG. 16) all have four-fold symmetry, the pentagon (FIG. 9) has five-fold symmetry, the hexagon (FIG. 10) has six-fold symmetry, and the octagon (FIG. 11) has eight-fold symmetry.

Note that the shape of the aperture may optionally include one or more curved portions, like a half-circle, or a flower-petal shape. The shape may optionally include one or more concave portions, like the corners of the cross in FIG. 13.

Note also that in some cases, the roles of the prime mover coupler 130 and the drive shaft coupler 160 may be reversed. In other words, a portion of the prime mover coupler 130 may fit inside a suitably shaped aperture on the drive shaft coupler 160, rather than the other way around.

Note that there may be gaps that exist between the coupler materials at the interface shown in FIG. 7-16. For instance, there may be a groove or notch cut out of the prime mover coupler 130 and/or the drive shaft coupler 160. There may even be multiple grooves or notches cut out at different longitudinal locations along the portion of overlap. Such gaps are permissible, as long as they do not significantly affect the rotational lockability of the two coupler parts when they are engaged.

In all cases, it is desirable that one coupler be able to slide longitudinally across (or within) the other coupler, while prohibiting rotation of one with respect to the other.

FIG. 17 is a cross-sectional schematic drawing of an atherectomy device 200, in which the longitudinal position of the drive shaft 150 is manually controllable by the operator. This feature may be useful in, for example, the following scenario. Consider a case where the guide wire has been advanced to, or past, a blockage, a catheter has been advanced over the guide wire to the blockage, a drive shaft has been advanced through the catheter over the guide wire so that the abrasive head at the distal end of the catheter is near the blockage, and the proximal end of the drive shaft has been attached to the prime mover.

In the atherectomy device 200 of FIG. 17, once the drive shaft 150 is rotationally locked to the prime mover 120 (i.e., if the prime mover 120 rotates, the drive shaft 150 is forced to rotate along with it), the longitudinal position of the drive shaft 150 is still controllable by the practitioner. This axial position control is an added feature, above and beyond all the advantageous features of the device 100 shown in FIGS. 5 and 6.

The handle on the device 200 of FIG. 17 includes a fixed handle portion 210 and an exchangeable handle portion 240, which may be similar in construction and function to those described above. In some cases, the handle portions may be separable and reattachable. In other cases, the handle may be sealed unit, which does not come apart. For a sealed unit, the drive shaft 150 may not be replaceable, but the device 200 still has the advantages of a strong rotational coupling between the prime mover 120 and the drive shaft 150, and an adjustable axial position of the drive shaft. Although the handle is shown in FIG. 17 as being two pieces, 210 and 240, it will be understood that these pieces may be constructed as a single unit for a sealed device.

The handle includes a controller or knob 280 that adjusts the longitudinal position of two opposing elements 285, which longitudinally surround a retaining element 265 on the drive shaft coupler 160. In some cases, the retaining element 265 is a ring extending laterally away from the rotational axis of the drive shaft 150, with the opposing elements 285 being plates that can push the ring longitudinally in the distal or proximal directions. The opposing elements 285 and retaining element 265 may be made from suitable materials that reduce friction during contact between them.

The catheter and drive shaft operate in a fluid environment, so it is beneficial to describe some of the plumbing used with the device.

Typically, a rotational atherectomy device sends a mixture of saline and a medical guide lubricant down the catheter, toward the blockage. The fluid helps protect the interior of the catheter and the exterior of the drive shaft, helps clear away material broken loose from the blockage, and helps equalize pressure in the vessel.

The fluid is preferably delivered into the catheter downstream from the couplers described above, rather than in the chamber that includes the keyed couplers. In some designs, a telescoping assembly may be added to the handle housing, which would be located to the right of the handle elements 140, 240 in FIGS. 5, 6 and 17. Such a telescoping assembly may be formed from two or more nested cylinders, such as two stainless or polyamide hypo tubes. The handle attaches to the proximal end of the telescope, and the distal end of the telescope attaches to an adapter that allows fluid to enter. In some cases, the adapter may be formed as a “T”, with the drive shaft 150 having a straight, axial path through the “T” and fluid entering from the lateral direction. The fluid may be delivered to the “T” adapter by a small, flexible tube that can be wrapped around the telescoping portion.

The telescoping portion itself primarily protects the drive shaft during use in two ways. First, it absorbs any axial motion that arises from the distal end of the device, thereby protecting the prime mover and the other moving parts in the handle. Second, the telescope restricts the motion of the drive shaft to lie on or very close to its own rotational axis. If the drive shaft were allowed to deviate too far from its own rotational axis, it may suffer damage due to large oscillations, analogous to the large lateral component in the motion of a jump rope.

Finally, we summarize much of the above disclosure by providing a description of a typical atherectomy procedure that uses the above-described device and method.

Initially, a guide wire is fed through the vasculature of a patient, and advanced through the vasculature until its distal end is at a particular blockage in a vessel. Preferably, the guide wire is fed until its distal end is just past the blockage, but this is possible only when the vessel is partially blocked by the blockage. Next, a catheter is advanced along the guide wire until its distal end is close to the blockage. The drive shaft is within the catheter and surrounds the guide wire. The drive shaft may be fed along with the catheter, or may be fed separately once the catheter is in place. The proximal end of the drive shaft is attached to the prime mover, using the keyed couplers described above. Such attachment may take place as the removable portion of the handle is attached to the fixed portion of the handle. The prime mover is powered up, causing the drive shaft to rotate, and causing an abrasive head at the distal end of the drive shaft to remove all or a portion of the blockage. The prime mover is powered down, and rotation stops. If the blockage is completely or sufficiently removed, then the catheter and drive shaft are withdrawn, and then the guide wire is withdrawn. If the blockage requires additional removal, the catheter and drive shaft are withdrawn, the removable portion of the handle is removed, and they are all replaced by a new removable portion, catheter, and drive shaft, with an abrasive head at the distal end of the drive shaft that can clear blockages to a larger diameter than the first abrasive head. In this manner, even if multiple catheters and drive shafts are used during a procedure for a particular patient, the prime mover and fixed portion of the handle may be reused, rather than discarded and replaced fresh for each new drive shaft. This saves a significant amount of cost, compared to discarding the entire assembly for each new drive shaft.

The description of the invention and its applications as set forth herein is illustrative and is not intended to limit the scope of the invention. Variations and modifications of the embodiments disclosed herein are possible, and practical alternatives to and equivalents of the various elements of the embodiments would be understood to those of ordinary skill in the art upon study of this patent document. These and other variations and modifications of the embodiments disclosed herein may be made without departing from the scope and spirit of the invention.

We claim:

1. A rotational atherectomy device, comprising:
a handle housing;
an elongated, flexible, rotatable drive shaft having a proximal end at the handle housing and a distal end opposite the proximal end for insertion into a vasculature of a patient;
a drive shaft coupler fixedly attached to the proximal end of the drive shaft and facing away from the drive shaft;
a prime mover within the handle housing for rotating the drive shaft; and
a prime mover coupler rotatably coupled to the prime mover and facing the drive shaft;
wherein the drive shaft coupler and the prime mover coupler have engageable lateral cross-sections that are complementary and are geometrically keyed; and
wherein engagement of the complementary lateral cross-sections allows axial translation between the drive shaft coupler and the prime mover coupler and prohibits rotational motion between the drive shaft coupler and the prime mover coupler.

2. The rotational atherectomy device of claim 1,
wherein the drive shaft and the drive shaft coupler are detachable from the handle housing and are replaceable; and
wherein the prime mover and the prime mover coupler are not detachable from the handle housing.

3. The rotational atherectomy device of claim 2, wherein the handle housing includes at least one mating feature for laterally aligning the drive shaft coupler when the drive shaft is replaced.

4. The rotational atherectomy device of claim 1, wherein the handle housing includes a mechanism for adjusting a longitudinal position of the drive shaft without adjusting a longitudinal position of the prime mover.

5. The rotational atherectomy device of claim 1, wherein the drive shaft coupler extends longitudinally through an aperture on the prime mover coupler, the extending portion of the drive shaft coupler having an exterior profile that rotationally locks within the aperture on the prime mover coupler.

6. The rotational atherectomy device of claim 1, wherein the prime mover coupler extends longitudinally through an aperture on the drive shaft coupler, the extending portion of the prime mover coupler having an exterior profile that rotationally locks within the aperture on the drive shaft coupler.

7. The rotational atherectomy device of claim 1, wherein the complementary lateral cross-sections of the drive shaft coupler and the prime mover coupler include a regular polygonal shape.

8. The rotational atherectomy device of claim 7, wherein the complementary lateral cross-sections of the drive shaft coupler and the prime mover coupler include a square.

9. The rotational atherectomy device of claim 7, wherein the complementary lateral cross-sections of the drive shaft coupler and the prime mover coupler include a hexagon.

10. The rotational atherectomy device of claim 1, wherein the complementary lateral cross-sections of the drive shaft coupler and the prime mover coupler include a shape having at least one rounded corner.

11. The rotational atherectomy device of claim 1, wherein the prime mover coupler is fixedly attached to the prime mover.

12. A method for removing a blockage from a vessel, comprising:
inserting a guide wire into a vasculature of a patient;
advancing the guide wire through the vasculature to the blockage;
removably attaching a proximal end of a first drive shaft to a prime mover, the attachment rotatably securing the first drive shaft to the prime mover and allowing longitudinal translation between the first drive shaft and the prime mover;
advancing the first drive shaft over the guide wire until a first abrasive head at a distal end of the first drive shaft is proximate the blockage;
rotating the first drive shaft;
partially removing the blockage in the vessel, the removal comprising a size determined by the first abrasive head; and
retracting the first drive shaft from the vasculature of the patient.

13. The method of claim 12, further comprising:
detaching the proximal end of the first drive shaft from the prime mover;
removably attaching a proximal end of a second drive shaft to the prime mover, the attachment rotatably securing the second drive shaft to the prime mover and allowing longitudinal translation between the second drive shaft and the prime mover;
advancing the second drive shaft over the guide wire until a second abrasive head at a distal end of the second drive shaft is proximate the blockage;
rotating the second drive shaft;
partially removing the blockage in the vessel, the removal comprising a size determined by the second abrasive head and different from the size determined by the first abrasive head;
retracting the second drive shaft from the vasculature of the patient; and
retracting the guide wire from the vasculature of the patient.

14. The method of claim 13,
wherein the insertion of the guide wire is performed before the advancing, rotating and retracting of the first and second drive shafts;
wherein the rotating of the first and second drive shafts is performed with the guide wire remaining in the vasculature of the patient; and
wherein the retracting of the guide wire is performed after the advancing, rotating and retracting of the first and second drive shafts.
